(19) United States
(12) Patent Application Publication
(10) Pub. No.: US 2006/0013858 A1
(43) Pub. Date: Jan. 19, 2006

(54) Fludrocortisone treatment for hearing loss

(76) Inventor: Dennis R. Trune, Tigard, OR (US)

Correspondence Address: KLARQUIST SPARKMAN, LLP, 121 SW SALMON STREET, SUITE 1600, PORTLAND, OR, 97204, US

(21) Appl. No.: 10/533,070

(22) PCT Filed: Oct. 29, 2003
(86) PCT No.: PCT/US03/34591

§ 371 Date: Apr. 28, 2005

Related U.S. Application Data
(60) Provisional application No. 60/422,470, filed on Oct. 29, 2002.

(51) Int. Cl. A61K 31/573; A61F 2/00

(52) U.S. Cl. 424/427; 514/179

(57) ABSTRACT

Methods and compositions for treating or stabilizing hearing loss due to cochlear disorders by administration of compositions that include fludrocortisone or a mimetic or analog thereof are disclosed.

### CROSS REFERENCE TO RELATED APPLICATION

This application claims the benefit of U.S. Patent No. 60/422,470 filed Oct. 29, 2002, herein incorporated by reference in its entirety.

### ACKNOWLEDGMENT OF GOVERNMENT SUPPORT

Some of the work described in this patent application was funded by NIH-NIDCD R01 DC03573, NIH-NIDCD R21 DC03955, NIH-NIDCD R01 DC05593, and VA RR&D National Center for Rehabilitative Auditory Research RCTR 597-0160, Portland VAMC. The government may have certain rights in this invention.

### FIELD

This disclosure relates to the use of fludrocortisone, as well as analogs and mimetics thereof, alone or in combination with other compounds, for the treatment or stabilization of hearing loss.

### BACKGROUND

Glucocorticoids have been traditionally used to reverse hearing loss in a variety of cochlear disorders. These include autoimmune and other systemic immune diseases (McCabe, Ann. Otol. 88, 585-9, 1979; Hughes et al., Immunologic disorders of the inner ear. In: Bailey, B. J. (Ed.), Head and Neck Surgery-Otolaryngology. Lippincott, Philadelphia, pp. 1833-41, 1993; Harris and Ryan, 1995), endolymphatic hydrops and Meniere's disease (Hughes et al., Laryngoscope, 93, 410-7, 1983; Dickens and Graham, Amer. J Otolaryngol., 11:51-65, 1990), and cases of idiopathic and sudden hearing loss when etiology is unclear (Moscowitz et al., Laryngoscope 94:664-6, 1984; Wilson et al., Arch. Otolaryngol. 106:772-6, 1980; O-Uchi et al, Auris-Nasus-Larynx (Tokyo) 20:79-93, 1993; Moscicki et al., JAMA, 272:611-6, 1994; Byl, Laryngoscope 94:647-61, 1984; Parnes et al., Laryngoscope Suppl 91:1-17, 1999). In spite of the effectiveness of glucocorticoids, their significant side effects prevent long term therapy and management of auditory and vestibular dysfunction (Sismanis et al., Otolaryngol. Head Neck Surg. 116:146-52, 1997).

Most glucocorticoids (except dexamethasone) have three physiological functions: anti-inflammation, immune suppression, and increased sodium transport/reabsorption. The rationale for glucocorticoid therapy for autoimmune and sudden hearing loss was traditionally based on the first two functions, that is, to counter presumed inflammation in the ear and suppress systemic immune processes. However, some have demonstrated that a major pathology in such idiopathic hearing loss is disruption of the cochlear stria vascularis and its blood-labyrinth barrier (Lin and Trune, OtolaryngoL Head NeckSurg. 117:530-4, 1997), which leads to decreased endocochlear potentials (Ruckenstein et al., Otolaryngol Head Neck Surg. 121:52-6, 1999).

The stria vascularis has numerous mineralocorticoid and glucocorticoid receptors (Rarey et al., Laryngoscope 101:1081-4, 1991; Lohuis et al., Acta Otolaryngol. 110:348-56, 1990; Rarey and Luttge, Hear. Res. 41:217-22, 1989; ten Cate et al., Laryngoscope 103:865-71, 1993). K⁺ is actively transported into the endolymph and Na⁺ out, all under the control of the Na⁺,K⁺-ATPase system that is regulated by circulating steroid levels (Rarey et al., Arch. Otolaryngol. Head Neck Surg. 115:817-21, 1989). Aldosterone increases the number of inner ear Na⁺,K⁺-ATPase binding sites (Pitovski et al., Brain Res. 601:273-8, 1993), and adrenalectomy removes circulating corticosteroids and results in edematous spaces in the stria (Lohuis et al., Acta Otolaryngol. 110:348-56, 1990), similar to the appearance in autoimmune disease.

Adrenalectomy-induced stria changes can be reversed with aldosterone (Rarey et al., Laryngoscope 101:1081-4, 1991). In addition, treatment of MRL/MpJ-Fas^{lPr} autoimmune mice with aldosterone, which increases sodium transport, was just as effective as prednisolone in reversing or stabilizing autoimmune related hearing loss (Trune et al., Laryngoscope, 110: 1902-6, 2000). However, aldosterone is not available for clinical use, because the body adjusts to administration of aldosterone by reducing production of more aldosterone. Therefore, it is difficult to obtain the serum levels necessary for treatment. Therefore, there is a need to identify a method to treat hearing loss due to defects in the stria vascularis using other compounds or agents that restore proper stria ion balances.

### SUMMARY

The inventor has identified a method of reversing or stabilizing hearing loss using the mineralocorticoid fludrocortisone (or mimetics or analogs thereof), alone or in combination with other compounds, which provides an unexpectedly superior effect to that of aldosterone. In addition, the use of fludrocortisone, instead of glucocorticoids such as prednisone (or in combination with lower amounts of such glucocorticoids) to treat or stabilize hearing loss will reduce the detrimental side effects commonly observed with glucocorticoids. In some examples, fludrocortisone treats a hearing loss due to a cochlear disorder by increasing Na⁺ and K⁺ transport to restore normal fluid ion balances in the stira vascularis and its blood-labyrinth barrier. In other examples, the hearing loss that is treated is an idiopathic hearing loss, for example a sudden sensorineural hearing loss.

Compositions that include therapeutically effective amounts of fludrocortisone (or mimetics or analogs thereof) and one or more glucocorticoids are also disclosed. The therapeutically effective amount of both the fludrocortisone and the glucocorticoids is lower than if either agent were administered alone. This reduces the incidence of undesirable side effects often observed with glucocorticoids.

### DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. As used herein and in the appended claims, the singular forms “a” or “an” or “the” include plural references unless the context clearly dictates otherwise. For example, reference to “a steroid” includes a plurality of such steroids and reference to “the mineralocorticoid” includes reference to one or more mineralocorticoids and equivalents thereof known to those skilled in the art, and so forth. Similarly, the word “or” is intended to include “and” unless the context clearly indicates otherwise. Hence “comprising A or B” means including A, or B, or A and B.

Unless explained otherwise, all technical and scientific terms used herein have the same meaning as commonly understood to one of ordinary skill in the art to which this disclosure belongs.

Analog: An agent (such as an organic chemical compound) that is structurally similar to another, but differs slightly in composition, for example the replacement of one atom by an atom of a different element or functional group. For example, an analog of fludrocortisone, such as fludrocortisone acetate, is structurally similar to fludrocortisone, and has a similar effect on treating hearing loss due to a cochlear disorder.

Cochlear disorder: A disease of the cochlea that results in some amount of sensorimeural hearing loss in a subject. Particular examples of such disorders result in disruption of the stria vascularis and its blood-labyrinth barrier. In some examples, a cochlear disorder decreases sodium transport in the stria vascularis and its blood-labyrinth barrier. A decrease in sodium transport is any amount that results in hearing loss, such as a decrease of at least 5%, at least 10%, at least 20%, at least 50% or even at least 90%, when compared to the sodium transport in a normal ear.

In some examples, hearing in the subject is reduced by about at least 10%, such as about at least 20%, such as about at least 50%, such as about at least 75%, or even such as about at least 90% or 100%. Non-limiting examples of cochlear disorders include autoimmune diseases (such as Wegener's granulomatosis, polyarteritis nodosa, Cogan's syndrome, rheumatoid arthritis, Sjogren's syndrome, and systemic lupus erythematosus), systemic immune diseases with antibodies against inner ear antigens, endolymphatic hydrops and Meniere's disease, and idiopathic rapid progressing and sudden hearing loss when etiology is unclear.

Comprises: A term that means “including.” For example, “comprising A or B” means including A or B, or both A and B, unless clearly indicated otherwise.

Fludrocortisone (9-a-fluorocortisol): A synthetic analog of aldosterone that acts on the kidney so as to conserve sodium and excrete potassium. Includes derivatives of fludrocortisone, such as fludrocortisone acetate (which goes by the brand name Florinef™; chemical structure 12-(acetyloxy)- -11β17α-dihydroxy-pregn-4-ene-3,20-dione), as well as mimetics and analogs thereof.

Glucocorticoids: Glucocorticoids are corticosteroid agents (including mimetics) that affect carbohydrate metabolism, and are involved in the suppressive control of the immune system and inflammation. Examples of glucocorticoids include, but are not limited to: hydrocortisone, dexamethasone, methylprednisolone, prednisone, and prednisolone. Hydrocortisone is a natural glucocorticoid while prednisone is a commonly prescribed synthetic glucocorticoid. In one example, glucocorticoids have as high of a binding affinity to the mineralocorticoid receptor as they do the glucocorticoid receptor, and therefore have mineralocorticoid activity. Prednisone is an example of a glucocorticoid that has also been found to display mineralocorticoid activity.

In one example, therapeutic glucocorticoids have one or more (such as one, two, or three) of the following functions: immune suppression, anti-inflammation, and sodium reabsorption. In a particular example, glucocorticoids can reverse autoinimune hearing loss by increasing strial sodium-potassium transport by a desired amount to restore normal endolymph ion balances.

Idiopathic hearing loss: A hearing loss for which there is no apparent cause (such as trauma). This type of hearing loss is often attributed to autoimmune or viral phenomena.

Mammal: This term includes both human and non-human mammals. Similarly, the terms “patient,” “subject,” and “individual” includes living multicellular vertebrate organisms, such as human and veterinary subjects.

Mimetic: A molecule (such as an organic chemical compound) that mimics the activity of a compound, such as the activity of fludrocortisone on hearing loss. Peptidomimetic and organomimetic embodiments are within the scope of this term, whereby the three-dimensional arrangement of the chemical constituents of such peptido- and organomimetics mimic the three-dimensional arrangement of the peptide backbone and component amino acid sidechains in the peptide, resulting in such peptido- and organomimetics of the peptides having substantial specific activity. For computer modeling applications, a pharmacophore is an idealized, three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs”, in Klegerman & Groves, eds., 1993, Pharmaceutical Biotechnology, Interpharm Press: Buffalo Grove, Ill., pp. 165-174 and Principles of Pharmacology (ed. Munson, 1995), chapter 102 for a description of techniques used in computer assisted drug design.

Mineralocorticoids: Corticosteroid agents (including mimetics) that have a role in electrolyte balance, achieved mainly through sodium reabsorption, such as in the kidney. Aldosterone is the main natural mineralocorticoid and fludrocortisone its synthetic analog. In some examples, a mineralocorticoid has substantially only mineralocorticoid activity, such as fludrocortisone, in contrast to other mineralocorticoids that do not substantially only have mineralocorticoid activity, such as prednisone.

Activation of the mineralocorticoid receptor occurs typically by the natural mineralocorticoid aldosterone or the natural glucocorticoid cortisol. This receptor activation results in the expression of multiple gene products called aldosterone-induced proteins (AIPs). These proteins increase sodium and potassium transport across the cell membrane by activation of existing sodium channels, synthesizing new ones, and increasing cellular Na⁺, K⁺-ATPase to drive the process. Although the primary action of mineralocorticoid receptor activation is to upregulate DNA transcription, there are also nongenomic responses that occur in less than 10 minutes, such as activating Na⁺-H⁺ antiporters, existing sodium channels, and Na⁺,K⁺-ATPases.

Normal ear: As used herein, refers to an ear that does not suffer from hearing loss, such as those due to cochlear disorders. In humans, normal hearing is defined as hearing thresholds at less than 25 dB. Mild hearing loss is threshold between 25-40 dB, moderate hearing loss is threshold between 40-70 dB, severe hearing loss is between 70-90 dB, and 90+dB threshold is profound hearing loss.

Sensorineural hearing loss: A loss of hearing (including partial or total deafness) due to a disorder of the sensory mechanism of the acoustic nerve or central nervous pathways. A cochlear sensorineural hearing loss is loss that is specific to the cochlea.

Sudden deafness: Severe sensorineural hearing loss that usually occurs in only one ear and develops over a period of a few hours or less, which is non-vascular in origin, and is due to an acute cochlear disorder. It is often idiopathic, in that there is no evident etiology, but is believed that the cause is often viral or autoimmune.

Therapeutically effective amount: An amount sufficient to achieve a desired biological effect, for example an amount that is effective to improve signs or symptoms of hearing loss due to a cochlear disorder, for example by increasing the ability of the subject to hear or preventing the subject's hearing from decreasing (that is, stabilizing hearing loss), or both.

In particular examples, it is a concentration of fludrocortisone or mimetic thereof, alone or in combination with other therapeutically effective agents, effective to reverse or stabilize cochlear dysfunction in a subject to whom it is administered, for example to treat or stabilize hearing loss. In additional or alternative examples, it is an amount of fludrocortisone or mimetic thereof effective to increase potassium or sodium transport in the stria vascularis by more than a desired amount, such as an increase by at least 10%, at least 20%, at least 50%, at least 75% or even at least 90% as compared to potassium and/or sodium transport prior to treatment. In other or additional examples, it is an amount effective to increase hearing in a subject suffering from hearing loss by more than a desired amount, such as increase by at least 10%, at least 20%, at least 50%, at least 75% or even at least 90% as compared to an amount of hearing in the ear of the subject prior to treatment.

An effective amount of fludrocortisone (or mimetic thereof) can be administered in a single dose, or in several doses, for example daily, during a course of treatment. However, the effective amount of fludrocortisone may be dependent on the source of fludrocortisone administered, the subject being treated, the severity and type of hearing loss being treated, and the manner of administration of fludrocortisone. For example, a therapeutically effective amount of fludrocortisone (or mimetic thereof) can vary from about 1 μg/kg body weight to about 20 μg/kg body weight per day, about 1 μg/kg body weight to about 10 μg/kg body weight per day, about 10 μg/kg body weight to about 20 μg/kg body weight per day, or about 1-2 μg fludrocortisone/kg body weight/day.

The therapeutically effective amount of fludrocortisone (or mimetic thereof) can be decreased when treatment of the subject includes another therapeutically effective agent, such as a glucocortiocid. For example, when a composition including fludrocortisone and prednisolone is administered to a subject, the therapeutically effective amount of fludrocortisone may be reduced to the range of 0.5-1.0 μg fludrocortisone/kg body weight (effective dose 0.0-0.2 mg fludrocortisone/day) and the therapeutically effective amount of prednisolone may be reduced to the range of 30-400 μg prednisolone/kg body weight (effective dose 2-30 mg prednisolone/day).

To assess the regression or stabilization of the hearing loss, the methods disclosed herein can be used to compare a subject before and after treatment. For example, inner ear function can be assessed by auditory brainstem response audiometry (example 2) and the endocochlear potential (Example 7); inner ear morphology can be assessed by light and electron microscopy as described in Examples 4 and 8; systemic autoimmune disease can be assessed by detection of serum immune complexes, hematocrits, and antinuclear antibodies as described in Example 3; and cochlear specific autoantibodies and upregulated gene products can be assessed with ELISA as described in Example 9 and 10.

### Treatment of Hearing Loss

Glucocorticoids have traditionally been used to treat hearing loss in a variety of cochlear disorders. However, in spite of glucocorticoid effectiveness, severe side effects such as increased susceptibility to infection, sodium and fluid retention, hyperglycemia, hypertension, muscle weakness, osteoporosis, increased ocular pressure, Cushingoid state, fat deposition (face), nervousness, and insomnia, prevent long-term management of inner ear dysfunction. Therefore, there is a need to identify agents that can correct hearing loss due to a cochlear disorder, but have fewer undesirable side effects.

It has now been found that many of the therapeutic benefits of glucocorticoids can instead be obtained with the mineralocorticoid fludrocortisone. Disclosed herein is a mechanism for reversal of autoimmune hearing loss by the restoration of proper stria ion balances (for example by increasing the reabsorption of sodium), mediated through the mineralocorticoid receptor. Stria ion balances do not necessarily need to be restored to 100% of normal. Any restoration of stria ion balances that improve the signs or symptoms of hearing loss, for example by increasing the ability of the subject to hear or stabilizing hearing loss is acceptable. In one example, the methods disclosed herein reverse or stabilize cochlear dysfunction by increasing K or Na transport to restore endolymph ion balances. In an additional or alternative example, administration of mineralocorticoids such as fludrocortisone (or mimetic or analog thereof) reestablish normal stria vascularis and cochlear function without the side effects observed with glucocorticoids like prednisone.

A method for treating or stabilizing hearing loss due to a sensorineural cochlear disorder in a subject by administering a composition that includes a therapeutically effective amount of fludrocortisone (or mimetic thereof or analog thereof) to the subject is disclosed. In one example, a clinical diagnosis is made to determine if a subject is one who would benefit from the methods disclosed herein. For example, such a diagnosis can be made in the appropriate clinical context. A candidate for this treatment may, for example, have experienced sudden hearing loss without an evident traumatic etiology. In addition, a determination can be made as to whether the subject has a viral or autoimmune disorder, in addition to the hearing loss, which would also be a clinical factor in favor of a condition suitable for treatment with the methods disclosed herein. Particular subjects with hearing loss that developed over a few hours or less, further indicates that the subject has a severe sensorineual hearing loss that may benefit from administration of a composition that includes fludrocortisone.

Hearing loss can result from any type cochlear disorder such as that seen in sudden or idiopathic hearing loss. The method is of particular use in treating such hearing loss characterized by decreased sodium transport in the stria vascularis and its blood-labyrinth barrier, for example due to an autoimmune disease that results in hearing loss, such as Wegener's granulomatosis, polyarteritis nodosa, Cogan's syndrome, rheumatoid arthritis, Sjogren's syndrome, or systemic lupus erythematosus. The subject can also suffer from a sudden hearing loss due to endolymphatic hydrops or Meniere's disease. The subject can be a mammal, such as a human or veterinary subject.

The subject in need of treatment can suffer from hearing loss in one or both ears. As a result, the amount of hearing loss observed in the subject will vary. In some examples, the subject has a reduction in hearing by at least 20%, when compared to the same subject before the hearing loss started, or when compared to “average” or “normal” hearing for others of the same sex and age, or when compared to the “average” hearing for the population as a whole. In other examples, the subject has a reduction in hearing by at least 30%, at least 40%, at least 50%, at least 60%, at least 70%, at least 80%, at least 90%, at least 95% or even 100% when compared to the same subject before the hearing loss started, or when compared to “average” hearing for others of the same sex and age, or when compared to the “average” hearing for the population as a whole. The percent reduction in hearing can be the amount of hearing reduced in only one ear, or the amount of overall hearing loss.

In some examples, administration of a composition that includes a therapeutically effective amount of fludrocortisone (or mimetic or analog thereof) decreases sodium-potassium imbalance in an endolymph of the stria vascularis of the treated subject. For example, sodium-potassium imbalance can be decreased by at least 10%, at least 20% at least 50% or even at least 75% in the endolymph of the stria vascularis. Administration of a composition that includes a therapeutically effective amount of fludrocortisone can also increase potassium and/or sodium transport in a stria vascularis of the treated subject. For example, potassium and/or sodium transport can be increased by at least 10%, at least 20% at least 50% or even at least 75% in the stria vascularis,

The amount of hearing restored by administration of fludrocortisone will vary among subjects. In some examples, hearing is not restored, but is instead stabilized so that additional substantive hearing loss does not occur. In another example, administration of a composition that includes a therapeutically effective amount of fludrocortisone increases hearing in the subject by at least 10%, such as at least 20%, at least 30%, at least 40%, at least 50%, at least 60%, at least 70%, at least 80%, at least 90%, at least 95% or even 100% when compared to the same subject before administration of the composition including fludrocortisone. The percent increase in hearing can be the amount of hearing increased in only one ear, or the amount of overall increased hearing. The methods disclosed herein will allow one to measure the structural (stria) and functional (endocochlear potential, EP) impacts of fludrocortisone treatment. This can be coupled with the cellular and molecular assessments of cochlear specific antibodies and upregulated gene products.

In some examples, the composition including fludrocortisone is administered with a pharmaceutically acceptable carrier. In additional or alternative examples, fludrocortisone is co-administered with a therapeutically effective amount of another therapeutic agent such as a glucocorticoid, for example prednisone. Co-administration can be simultaneous, or one following the other, such as within a few minutes, or within a few hours, such as within 1 or 2 hours. When fludrocortisone is co-administered with a glucocorticoid, lower doses of each compound are more effective (and with fewer side effects) than either alone.

Any mode of administration can be used, as long as the method is effective to deliver the active agent to the site of action (the cochlea). Modes of administration include, but are not limited to oral administration, transdermal administration (such as near the ear), administration directly to the ear (intraotically, intratympanically, or transtympanically), such as with ear drops which include fludrocortisone at a therapeutically effective dose or injection into the middle ear.

### EXAMPLE 1

### Administration of Steroids to MRL/MpJ-Fas^{lPr} Autoimmune Mice

The MRL/MpJ-Fas^{lPr} autoimmune mouse is an established model of autoimmune sensorineural hearing loss (Ruckenstein et al., Acta OtolaryngoL 113:160-5, 1993; Trune et al. Hear. Res. 105:57-64, 1997). These mice carry a Fas gene defect that prevents apoptosis of self-recognizing T lymphocytes, leading to T cell proliferation and polyclonal B cell activation (Watanabe-Fukunaga et al. Nature 356:314-7, 1992). Spontaneous systemic autoimmune disease develops in MRL/MpJ-Fas^{lPr} autoimmune mice, resulting in increased serum immune complexes, anti-DNA antibodies, lymphadenopathy, elevated auditory thresholds, lowered hematocrits due to anti-erythrocyte autoantibodies, splenomegaly, increased body mass and pathological changes in the stria vascularis (Id. and Ruckenstein et al. Otolaryngol. Head Neck Surg. 121:452-6, 1999; Trune et al. Hear. Res. 155:9-20, 2001).

Mice (Jackson Laboratories, Bar Harbor, Me.) were obtained at two months of age. Onset of systemic autoimmune disease occurs at 3-4 months of age and cochlear thresholds rise shortly thereafter (Trune et al., Otolaryngol. Head Neck Surg. 117:504-8, 1997). Mice were tested with auditory brainstem response (ABR) audiometry (see Example 2) at 2-3 months of age to establish pretreatment baseline auditory thresholds.

Serum samples were collected for baseline levels of hematocrit, serum immune complexes, and antinuclear antibodies, all hallmarks of systemic autoimmune disease. Following these determinations, mice were randomly assigned to steroid treatment groups (such as aldosterone, prednisolone, fludrocortisone, or combinations thereof) or water groups, for two months of treatment. At the end of treatments, ABR audiometry and serum collection were repeated at 2, 3, and 4 months of treatment to determine steroid effects on cochlear function and systemic autoimmunity.

Steroids, such as fludrocortisone, can be administered by any method used by those skilled in the art, such as oral, intraotically (see Example 13) or iv. Oral delivery in mice provides a constant source of steroid, parallels oral administration in humans, and avoids the trauma of daily injections. Mice drink 3-5 ml of water daily, so the effective dose for each treatment can be estimated. For example, the effective dose of steroid, such as fludrocortisone, can be from about 0.15 μg/day to about 0.30 mg/day.

Mice receiving glucocorticoid were administered a daily oral dose (1, 3, 5 or 10 mg/kg/day) of prednisolone sodium phosphate (Spectrum Quality Products, Inc., Gardena, Calif.), The steroid was provided orally by dissolving it in a standard 500 ml drinking water bottle, which suitably maintains elevated systemic levels (Zhou et al., Int. J. Immunopharm. 16:845-54, 1994; Hunnyball et al., Agents Actions 18:384-93, 1986; van der Kraan et al., Ann. Rheum. Dis. 52:734-41, 1993).

Mice receiving aldosterone were administered a daily oral dose of 3, 5, 15, or 30 μLg/kg/day of d-aldosterone (Sigma, St. Louis Mo.). Aldosterone drinking water was prepared by dissolving 50 μg of steroid in 50 μl of 100% ETOH, then diluting the required amount in the 500 ml of water in the drinking bottle to reach the final effective dose. The highest final ETOH concentration in a waterbottle was 0.018% (90 μl dose) and was considered negligible.

Mice receiving fludrocortisone were administered a daily oral dose of 3 μg/kg per day or 10 μg/kg per day. Fludrocortisone drinking water was prepared by dissolving 50 μg of fludrocortisone, 50 μl of 100% ETOH, then diluting the required amount in the 500 ml of water in the drinking bottle to reach the final effective dose. The final ETOH concentration in a water bottle was considered negligible.

Untreated control MRL/MpJ-Fas^{lPr} autoimmune mice were administered tap water to assess the normal progression of auditory dysfunction with systemic autoimmune disease.

Mice tolerate prednisolone and aldosterone in the drinking water and show no adverse affects, such as dehydration and avoidance of drinking. Mice similarly tolerate fludrocortisone. By the end of the two months of treatment, half of the untreated control mice will likely die of disease, which is typical for this strain. Survival is likely be statistically higher for mice on the steroid treatments when compared to untreated controls. The prednisolone groups show about 60-65% survival (p<0.05), while the aldosterone and fludrocortisone treatments show 60-80% survival (P<0.001).

### EXAMPLE 2

### Cochlear Function

This example illustrates that auditory brainstem response (ABR) audiometry to pure tones can be used to evaluate cochlear function using the method of Mitchell et al. (Hear. Res. 99:3846, 1996). Animals were anesthetized (kcetamine+xylazine) and the individual ears of each mouse stimulated with a closed-tube sound delivery system sealed into the ear canal. The ABR to tone-burst stimuli at 4, 8, 16, and 32 kHz was recorded and thresholds obtained for each ear. Absolute thresholds and threshold changes over the treatment period for each ear were calculated to determine if treatments impact cochlear function. For each ear, pretreatment and posttreatment thresholds at the four frequencies were compared statistically to establish any change over the treatment period.

The shifts for each frequency within an ear were added to derive its total change in threshold as described previously (Trune et al., Hear. Res. 137:160-6, 1999; Trune et al., Hear. Res. 137:167-73, 1999). If the ear's combined posttreatment thresholds was lower by 20 dB or more, the ear was considered to be improved (average of 5 dB per frequency). The ear is considered unchanged if the combined thresholds are +/−15 dB and worse if the combined thresholds were higher by 20 dB or more. Chi-square analyses was performed on the number of ears within each outcome category for each steroid treatment relative to water controls.

The shift in thresholds between baseline and posttreatment times was analyzed for each ear with a paired t-test. The number of ears better, unchanged, or worse over the treatment periods was compared with the treatment groups by means of the Chi-squared (X²) statistic to determine if any steroid treatment combination significantly altered the progression of hearing loss. The X² test also was also used to determine if steroid treatment significantly affects survival. A probability value less than 0.05 was considered significant in all tests. One advantage of this method is that nonsurviving mice do not bias the statistics. In addition, by summing the shifts at each frequency, any random fluctuations in thresholds at the different frequencies due to equipment or animal variation are mathematically removed.

It has been previously shown that thresholds in untreated (water control) autoimmune mice continue to increase with advancing systemic disease. This was particularly evident at the higher frequencies tested where thresholds increased 10-20 dB over the two month treatment period. Paired t-tests of the pretreatment and posttreatment thresholds for each ear showed significant elevations at 4, 16, and 32 kHz (P<0.05). On the other hand, steroid treatments (prednisolone or aldosterone) prevent significant overall threshold changes as average thresholds in these groups were similar to pretreatment baselines. Following 2 months of treatment, similar or better results were seen when using fludrocortisone as compared to aldosterone (p<0.001) (Table 1).

TABLE 1
Two months following treatment: %
of ears that are better-same-worse

| | # ears | better | same | worse |
|---|---|---|---|---|
| Fludrocortisone | 44 | 11.5 | 54.5 | 34 |
| Aldosterone | 26 | 4 | 27 | 69 |
| water | 38 | 8 | 39 | 53 |

If the variability in threshold changes is high due to the fact that some mice were better, some worse, and some unchanged from baseline, to establish a more clear picture of individual ear treatment effects, the pretreatment and posttreatment threshold differences at each frequency are summed to classify an ear as better, worse, or unchanged. After the two-month period, 78% of the ears in surviving water control mice were worse by 20 dB or more. Only two ears (11%) showed improvement by more than 20 dB and the remaining two ears were unchanged. The prednisolone and aldosterone-treated ears had significantly better thresholds than controls after two months of treatment. Consistently only 9-20% of the ears were worse with any of the prednisolone or aldosterone treatments. Across all prednisolone and aldosterone doses, 20-40% were better and 40-60% were unchanged. Fludrocortisone treatment led to 12% better and 55% unchanged, which was markedly and surprisingly better than the 4% better and 27% unchanged for aldosterone. The combination of aldosterone and prednisolone showed 10% better and 58% unchanged. When Chi-squared analyses were performed on threshold shifts for each dose relative to water controls, all treatments caused statistically better cochlear function.

To assess cochlear function in humans, an audiogram can be administered using standard methods used by those skilled in the art. In addition, an Audioscan (a form of high definition audiometry based on iso-hearing level frequency sweeps) can be performed using standard methods (for example, Zhao et al., Clin. Otolaryngol. 27(1):4-10, 2002).

### EXAMPLE 3

### Systemic Immune Disease

This example describes methods used to measure the severity of systemic autoimmune disease, as described in Trune et al. (Hear. Res. 38:57-66, 1989). Briefly, baseline and posttreatment serum samples are taken for measurement of hematocrit, antinuclear antibodies, and serum immune complexes as indices of systemic autoimmune disease progression. Although this example describes methods for testing mice treated with aldosterone, similar methods can be used to measure the severity of systemic autoimmune disease in humans, before and after treatment with fludrocortisone. For those mice still alive at the time of sacrifice, some spleens were weighed to assess splenomegaly, another index of systemic autoimmune disease progression.

Treatment with aldosterone or prednisolone affect various serum and organ measures. It may not be possible to perform an extensive statistical analysis on serum measures if there are high mortality rates, if the differential mortality rates between groups lead to large differences in sample sizes, and an inability to get sufficient blood from some mice. In this case, pretreatment-posttreatment statistical comparisons (paired t-tests) are conducted only for those mice that survive the treatment period.

Blood hematocrit in normal mice is approximately 45%. Autoimmune disease lowers the hematocrit due to anti-red blood cell autoantibodies, as demonstrated by the hematocrit of 40% in the untreated autoimmune mice. Prednisolone elevated hematocrits, presumably due to its immune suppression actions. Mice at the lower prednisolone dose had average hematocrits in the normal range and the pre-posttreatment comparison showed no significant difference (P=0.25). On the other hand, the higher dose prednisolone elevated hematocrits beyond the normal range (P=0.003). Aldosterone, which has no immune suppression function, did not cause any significant change in hematocrit at any dose. Fludrocortisone has minimal immune suppression function, and did not result in any significant change in hematocrit.

The level of serum immune complexes is related to the status of autoimmune disease. The normal levels of serum immune complexes is 25-100 μg/ml. In autoimmune mice, these levels can reach several thousand μg/ml, as demonstrated by the 6-7,000 μg/ml average in the untreated autoimmune group. Treatment with prednisolone, which has immune suppression functions, lowered serum immune complexes in a dose dependent manner. The 5 mg dose reduced immune complexes from 4,570 μg/ml to 1,092 μg/ml (P=0.0003). The higher prednisolone dose dropped serum immune complex levels from 3,920 μg/ml to approximately 450 μg/ml (P=0.012), which is close to normal. Those mice treated with aldosterone did not show any significant depression of serum immune complexes with treatment, presumably due to the fact that aldosterone has no immune suppression function. Mice treated with the higher dose aldosterone had posttreatment levels of 53,000 μg/ml, due to extremely high levels (157,000 μg/ml and 45,000 μg/ml) in two of the four surviving mice for which serum was available. Fludrocortisone, like aldosterone has minimal immune suppression function, and does not result in any significant change in serum immune complexes.

Antibodies against nuclear material are another manifestation of autoimmune disease. Anti-nuclear antibody (ANA) levels are determined by level of immunofluorescence (1+ to 4+) with normal as 1+as previously described (Trune et al., Hear. Res. 38:57-66, 1989). Comparison of pre- and posttreatment levels indicated that aldosterone and prednisolone treatment did not have a significant impact on the continuing elevation of ANA levels with progression of the disease.

Increased spleen size also is a manifestation of autoimmune disease, increasing with progression of systemic T and B cell proliferation. The spleen is approximately 75-100 mg in normal mice. An analysis of variance of all groups showed a significant group difference in spleen weights (F=4.28, P=0.034). Group comparisons showed the untreated autoimmune mice had an average spleen weight of 295 mg, while mice given the 5 mg prednisolone treatment had spleens of 45 mg, within the normal range (P=0.005). Spleens of the aldosterone mice showed no effect of steroid treatment and had average spleen weights of 399 mg (P=0.320), typical for autoimmune disease.

To determine the severity of autoimmune disease in humans, serum analysis is performed to detect for the presence or absence of immune complexes, anti-DNA antibodies, and/or rheumatoid factor, wherein the presence of such agents is indicative of the severity of autoimmune disease in a particular subject.

### EXAMPLE 4

### Cochlear Histology

This example describes methods used to quantitatively measure cochlear morphology changes resulting from steroid therapy, for example changes in the stria affected in autoimmune inner ear disease.

Following steroid treatment (or not, for control mice), the inner ears of some surviving mice were removed, perilymphatically perfused with fixative (3% paraformaldehyde in 0.1M phosphate buffer), and immersion fixed overnight. Following decalcification in EDTA, the ears were cryostat sectioned for qualitative morphologic evaluations of the stria vascularis. The procedure of Whitlon et al. (Brain Res. Protocols, 6:159-66, 2001) can be used to improve OCT compound infiltration and tissue preservation. The cochleas were cryostat sectioned for immunocytochemical analyses.

Quantitative morphology was performed on glycol methacrylate (GMA) embedded tissue as follows. Animals were perfused with fixative (3% paraformaldehyde in 0.1M phosphate buffer) and inner ears removed and immersion fixed overnight. Following decalcification in EDTA, the ears were embedded in glycol methacrylate for light microscopic examination. Every fourth section (5 μm thickness) wass serially mounted on glass slides and stained for quantitative and qualitative analyses. Cochleas were scanned for pathological changes and treatment effects. Of particular interest is the stria vascularis, lateral wall, hair cells, spiral ganglion neurons, and all blood vessels in modiolar and sensory regions. The area occupied by stria vascularis, blood vessels, non-blood vessels, and intercellular edema was measured for statistical analysis.

Alternatively, the following methods can be used to qualitatively determine stria vascularis and spiral ligament and integrity of endothelial cell tight junctions, intercellular edema. Mice are perfused with fixative (1.5% glutaraldehyde, 3% paraformaldehyde in 0.1M phosphate buffer), and the inner ears removed, decalcified in EDTA, and embedded in Araldite. Thin sections are observed on a Philips CM100. Qualitative observations are made of the cochlear tissue areas above and any other regions determined in the immunohistochemistry section to be responsive to steroid therapy. Stria vascularis is examined for integrity of endothelial cell tight junctions, and intercellular edema, to determine the treatment effects on known changes that occur in autoimmune disease.

The consistent cochlear pathology in untreated autoimmune mice was the degeneration of the stria vascularis, demonstrated as dilation of blood vessels, edematous intercellular spaces, and thinning of the stria as disease progressed. The general impact of steroid treatment was to restore the stria epithelium to a more normal appearance. Prednisolone treatment resulted in strias that showed the proper epithelium thickness, although dilated vessels still were seen. Qualitatively, the prednisolone stria appeared similar to younger autoimmune mice, prior to significant systemic autoimmune disease. This parallels the ABR data showing thresholds in most prednisolone mice either improved or did not get worse from baseline.

Aldosterone consistently restored the stria to almost normal appearance. The blood vessels were reduced to normal diameter, the edematous spaces were absent, and the epithelium was of normal thickness. The best results for aldosterone were seen with the higher doses. The lowest dose mice had stria epithelia with some edema and large vessels, while strias in the 15 and 30 μg treatment groups had strias that were virtually normal in appearance. This indicates that stria morphology improvement was related to the degree of sodium transport restoration.

To assess cochlear morphology in humans before and after treatment with fludrocortisone alone or with another agent such as prenisolone, an audiogram can be performed. In addition, gadolinium-enhanced magnetic resonance imaging (GdMRI) can be used to asses cochlear pathology associated with hearing loss (for example, see Hegarty et al., Laryngoscope 112(1):8-17, 2002).

### EXAMPLE 5

### Effect of Mineralocorticoid Receptor Antagonist

As described above, MRL/MpJ-Fas^{lpr} autoimmune mice treated with aldosterone have hearing improvement equal to those treated with prednisolone. This indicates that the restoration of hearing with steroids was due to an effect on sodium transport rather than an anti-inflammatory or immunosuppressive role. To demonstrate that corticosteroids reverse autoimmune hearing loss via the mineralocorticoid receptor, and that blocking the mineralocorticoid receptor will prevent glucocorticoid effects, the following methods were used.

Spironolactone, a mineralocorticoid receptor antagonist, was administered to MRL/MpJ-Fas^{lpr} autoimmune mice alone or in combination with corticosteroids in the drinking water as described above in Example 1, to block the hearing preservation observed with the administration of both glucocorticoids and mineralocorticoids. The four treatment groups were: spironolactone, spironolactone+aldosterone, spironolactone+prednisolone, and untreated water controls. More animals can be assigned to the water control group to allow for predicted poorer survival. The spironolactone treatment group was given daily oral doses (5 mg/kg per day) of spironolactone (Sigma, St. Louis, Mo.). Spironolactone drinking water was prepared by dissolving 15 mg of spironolactone in 0.6-0.8 ml of 100% ethyl alcohol (ETOH) and diluting it into a 500 ml drinking-water bottle. Mice drink 3-5 ml of water daily, so the effective dose was approximately 0.15 mg per day. The final ETOH concentration was approximately 0.1% and considered negligible. The dose of spironolactone administered (5 mg/kg per day) was equivalent to the dose used clinically for the treatment of primary hyperaldosteronism and was likely insufficient to completely block the mineralocorticoid receptor.

The spironolactone+aldosterone treatment group was given 15 μg/kg per day of aldosterone in addition to the 5 mg/kg/day of spironolactone. Aldosterone drinking water was prepared by dissolving 50 μg of aldosterone in 50 μl of 100% ETOH and diluting it in the same 500 ml drinking-water bottle as the spironolactone. The final effective dose of aldosterone was approximately 0.5 μg per day. The final ETOH concentration of aldosterone was less than 0.009%. The combined ETOH concentration of both compounds was less than 0.11% (0.1%+0.009%) and considered negligible.

The spironolactone+prednisolone treatment group was given daily oral doses (5 mg/kg per day) of prednisolone sodium phosphate in addition to the 5 mg/kg/day of spironolactone. The steroid was delivered by dissolving 15 mg in the same 500 ml drinking-water bottle as the spironolactone. The effective dose was approximately 0.15 mg per day.

The greatest alcohol concentration given was 0.16%. To determine if this amount of alcohol solvent had any impact on hearing and autoimmune disease, a control group of mice was given 750 μl of ETOH in the 500 ml drinking bottle for a final concentration of 0.15% and a final effective dose of 7.5 μl per day.

Mice tolerated spironolactone and steroid drinking water without obvious adverse effects, such as dehydration. At all treatment times, all groups showed similar attrition rates and there was no statistically significant difference in survival among the groups (P>0.05). However, approximately 50% of the mice died within the three-month period so that all hearing analyses were conducted on survivors, which were likely the least diseased mice.

ABR thresholds were recorded before and during treatment (2, 3, and 4 months) to measure the effect of steroids on hearing decline as described above in Example 2. As expected, cochlear function progressively declined in untreated mice over the four-month treatment period. The threshold elevation was most pronounced in the higher frequencies, which is typical for the autoimmune mice. Similar results were observed in the spironolactone and spironolactone+prednisolone mice, which showed little threshold change over the treatment period.

Ears also were analyzed independently across all frequencies. For each ear, the baseline and treatment ABR thresholds at the four frequencies were compared to establish any change attributable to treatment. The shifts at each frequency within an ear were added to derive a total shift in threshold per ear. The ear was considered to be improved, unchanged or worse as described in Example 2. The X² analyses compared the number of ears within each outcome category for each steroid relative to controls receiving water.

The ABR threshold analysis at two months of treatment (4-5 months of age) indicated that thresholds in untreated mice were predominately unchanged or worse. Only 29% control ears were improved and 25% were worse. Similar results were observed for the various steroid-treated animals, with the majority of ears remaining unchanged or worse. There was a trend toward worse hearing results in the spironolactone+prednisolone mice and better hearing results in the spironolactone+aldosterone mice, but these did not reach statistical significance when compared to water controls.

At three months of treatment 56% of the mice remained. Eight of ten spironolactone+aldosterone mice were still alive, whereas all other groups had lost approximately 50%. The ABR analysis demonstrate that thresholds in untreated mice continued to rise with advancing systemic disease and 31% of ears in water control mice were worse. The mice treated with spironolactone alone were no different from untreated mice in their hearing thresholds (P=0.56). However, at this time period the steroid-treated ears began showing different patterns of hearing. The spironolactone+prednisolone mice were significantly worse than the water controls (P=0.029) and the spironolactone+aldosterone ears were better than water controls (P=0.02). The comparison of these two combination groups indicated that those receiving aldosterone remained largely unchanged, while those receiving prednisolone progressively lost hearing, leading to a significant difference in treatment effects between these two groups (P=1.47e⁻⁸).

After four months of treatment many mice died of disease, leaving about 30-40% of mice alive in each group. The only exception was the 70% survival observed in mice receiving spironolactone+aldosterone. Although the hearing evaluations were made on only a few remaining ears, similar trends in hearing loss were observed. Hearing continued to decline in untreated mice with 50% of ears being worse than baseline. Mice receiving spironolactone alone showed no difference from controls. Seven of the eight remaining ears in the spironolactone+prednisolone mice were worse than baseline, but this did not reach statistical significance (P=0.101). The spironolactone+aldosterone mice still showed hearing patterns significantly better than water controls (P=0.037), with only 2 of 13 ears worse than they were at baseline. Thus, hearing in these mice did not decline significantly from baseline over the four-month treatment period. As with the other treatment periods, the spironolactone+prednisolone mice were significantly worse than the spironolactone+aldosterone mice (P=3.17e⁻²⁶).

Although spironolactone had a significant impact on hearing by blocking the mineralocorticoid receptor, it did not block the glucocorticoid receptor mediated effects on systemic immune disease. Mice given prednisolone, whether alone or in combination with spironolactone, still showed all of the normal immune suppression effects of the glucocorticoid. Immune complexes (total immunoglobulin) were lower and hematocrits and body weights remained normal. In contrast, mice receiving water, spironolactone alone, spironolactone+aldosterone, or ethanol all showed normal progression of systemic disease. Immune complexes and body weights were significantly higher and hematocrits were significantly lower.

In summary, spironolactone competitively blocked glucocorticoid-mediated hearing preservation in MRLIMpJ-Fas^{lPr} autoimmune mice. Supplemental aldosterone, by having a higher affinity for the mineralocorticoid receptor than spironolactone, was sufficient to override the spironolactone effects. This indicates that the mineralocorticoid receptor is the therapeutic target of corticosteroids in the reversal of autoimmune and sudden sensorineural hearing loss. Pharmacological treatments that selectively bind the mineralocorticoid receptor can provide maximal auditory benefit with fewer systemic side effects in patients with autoimmune sensorineural hearing loss.

### EXAMPLE 6

### Drug Combination Therapy

This example describes methods used to treat hearing loss using combination steroid therapy; mineralocorticoids to restore cochlear function and glucocorticoids to control systemic autoimmune disease. Because two drugs in combination can be more potent than when given alone, lower therapeutic levels of each were administered. One advantage of this method is that the negative side effects of the glucocorticoids can be reduced or eliminated if the glucocorticoid is administered in lower amounts.

MRIMpJ-Fas^{lpr} autoimmune mice were administered aldosterone (3, 5 or 10 μg/kg/day) or prednisolone (1 or 3 mg/kg/day) in their drinking water as described in Example 1, to establish the lowest effective therapeutically effective amount of each when administered alone. Hearing loss was monitored monthly using the method described in Example 2. Other groups of MRL/MpJ-Fas^{lpr} mice received combinations of aldosterone and prednisolone as follows: 0.5 mg/kg/day prednisolone+1.5 μg/kg/day aldosterone; 1 mg/kg/day prednisolone+3 μg/kg/day aldosterone; or 1.5 mg/kg/day prednisolone+5 μg/kg/day aldosterone.

Hearing loss was not prevented, nor did the rate of hearing loss decrease wit prednisolone alone at 1 mg/kg/day or by aldosterone alone at 3 μg/kg/day (Table 2) However, these doses did effectively control hearing loss when administered together (Table 2).

TABLE 2
Results for ears after 2 months of treatment

| | # (%) | # (%) no | # (%) | | # Mice | Alive at 2 | |
|---|---|---|---|---|---|---|---|
| | Worse | change | Better | Total | began tx | months of Tx | % alive |
| Aldosterone 10 μg/kg/day | 13 | | | | | | |
| | (93%) | 1 (7%) | 0 | 14 | 8 | 7 | 87.5% |
| Prednisolone 1.5 mg/kg/day + | 24 | | | | | | |
| Aldosterone 5 μg/kg/day | (80%) | 6 (20%) | 0 | 30 | 24 | 15 | 62.5% |
| Prednisolone 3 mg/kg/day | 6 | | | | | | |
| | (100%) | 0 | 0 | 6 | 7 | 3 | 42.9% |
| Prednisolone 3 mg/kg/day + | 10 | | | | | | |
| Aldosterone 10 μg/kg/day | (32%) | 18 (58%) | 3 (10%) | 31 | 22 | 16 | 72.7% |
| Prednisolone 5 mg/kg/day | 12 | | | | | | |
| Aldosterone 15 μg/kg/day | (54.5%) | 11 (44%) | 2 (8%) | 25 | 22 | 13 | 59.1% |
| Water | 18 | 6 | 1 | 25 | 42 | 13 | 31.0% |

Calculation of combination treatment on survival

| Survival | P1.5A5 | P3A10 | P5A15 | | | |
|---|---|---|---|---|---|---|
| Alive | 15 | 16 | 13 | 44 | 21.6104 | Chi sq |
| water expected | 9.3 | 6.8 | 6.8 | 22.9 | 2E−05 | Prob |
| chi sq calculation | 3.4935 | 12.447 | 5.653 | 21.5935 | | |

Similarly, a glucocorticoid and fludrocortisone can be co-administered to treat or stabilize hearing loss in a subject (see Example 12). For example, 10 prednisolone can be administered at a dose of 0.03-0.4 mg/kg/day for an effective dose of 2.5-40 mg/day and fludrocortisone can be administered at 0.5-1.0 μg/kg/day for an effective dose of 0.1-0.2 μg/day).

### EXAMPLE 7

### 15 Measurement of Endocochlear Potential

This example describes methods used to measure the endocochlear potential (EP) as a direct electrophysiologic manifestation of stria dysfunction. To measure directly the impact of systemic autoimmune disease on stria function, the EP in young and old autoimmune mice and age matched older BALB/c mice was measured.

While under ketamine+xylazine anesthesia, the animal's head was firmly fixed using a custom-made head holder on a three-dimensional positioning stage. A tracheotomy was performed to ensure free breathing and rectal temperature is maintained at 38.1° C. with a servo-regulated heating blanket. The bulla was exposed using a ventral approach through the same surgical field for the tracheotomy. The bony wall in front of the round window niche was removed and the round window membrane exposed. A glass micro-electrode with a tip diameter of approximately 0.5 μm and filled with 300 mM KCl was inserted through the round window membrane and the basilar membrane. A silver/silver chloride electrode inserted in the soft tissue of the neck served as a reference electrode. A BMA-200 bioamplifier with a super-Z head-stage (CWE, Inc. Ardmore, Pa.) was used to amplify the signal by a gain of 10. The voltage output of the amplifier was read using a digital multimeter. The zero level was established by positioning the baseline while the tip of the glass electrode is in the scala tympani, immediately before it penetrates the basilar membrane. When the tip of the microelectrode is advanced toward the scala media, the stable DC voltage following a sudden voltage increase is considered to be the EP.

The young autoimmune mice, prior to disease onset, show normal EPs, as do the 7 month old BALB/c mice (average value of 110 mV). This reflects the normal hearing levels of BALB/c mice at this age. The older autoimmune mice averaged only 74.8 mV. However, they were distributed in two distinct populations, a group with EPs below 70 mV and another population with normal EPs above 100 mV. This demonstrated the significant drop in stria function that occurs in the progression of autoimmune inner ear disease.

An EP measurement can be used to directly assess stria vascularis function and its control by steroid treatments, such as fludrocortisone. In humans, EP measurements would not likely be made, but instead an audiogram could be performed to assess stria vascularis function and its control by steroid treatments.

### EXAMPLE 8

### Stria Morphology Changes Due to Steroid Treatment

To increase histological detail in order to quantitatively measure steroid responsive hearing loss (stria blood vessel sizes), plastic embedding methods were used as described herein. Age-matched BALB/c controls also were processed to provide details of non-autoimmune stria. Camera lucida drawings were made of the stria, its blood vessels, and the edematous spaces. Digitized measurements were made of these various features. The most noticeable difference was seen in the larger size of the blood vessels in the untreated autoimmune mice. This paralleled previously studies that showed breakdown of vascular integrity in the stria vascularis with progressing disease (Trune, Otolaryngol. Head Neck Surg. 117, 504-8, 1997; Lin and Trune, Otolaryngol. Head Neck Surg. 117, 530-4, 1997).

The median vessel size in BALB/c mice was 35.3 μm², with 50% of vessels being larger and 50% being smaller in diameter. The number of blood vessels above and below the normal median was calculated for the autoimmune mice receiving water, prednisolone, and aldosterone. The results demonstrate that mice receiving only water (normal progression of autoimmune and cochlear disease), had much larger vessels than BALB/c controls (X²=10.31; p=0.0058). Mice who received prednisolone had blood vessels larger than, but close to, normal (X²=6.27; p=0.043). Mice who received aldosterone had blood vessels the same size as normal mice (X²=4.82 p=0.09), demonstrating the aldosterone treatment prevented the stria from developing the autoimmune pathology.

### EXAMPLE 9

### Steroid Impact on Cochlear-Specific Autoantibodies

The methods disclosed in this example were used to measure anti-cochlear autoantibodies in a subject having an autoimmune disease. The primary systemic impact of autoimmune disease is the elevation of circulating autoantibodies. These autoantibodies may affect the ear because of the presence of several antigens recognized by serum antibodies of patients with autoimmune ear disease, sudden or rapidly progressing hearing loss, and Meniere's disease. These include, but are not limited to, antigens such as heat shock protein 70, collagen type II, endothelial cells, cardiolipin, and laminin. Identified antigen-antibody reactions upon treatment with steroids, such as fludrocortisone, can reduce the levels of these putative autoantibodies.

To demonstrate the relationship of autoantibodies and ear disease, the reactivity of serum antibodies in the MRLIMpJ-Fas^{lpr} autoimmune mouse model for reactivity against the numerous proposed autoantigens for clinical hearing loss was determined as described below. Similar methods can be used to measure anti-cochlear autoantibodies in a human subject having an autoimmune disease before and after treatment with one or more steroids, such as fludrocortisone.

Sera were collected from normal C3H mice and MRLIMpJ-Fas^{lpr} autoimmune mice with 20-40 dB hearing loss. Mouse sera were tested for reactivity against putative cochlear antigens (laminin, heparan sulfate proteoglycan, cardiolipin, collagens II and IV, and three sources of heat shock protein 70 (bovine brain, mycobacterium, human recombinant)) by a standard enzyme-linked immunoabsorbance assay (ELISA) previously described (Hefeneider et al., Autoimmun. 15:187-94, 1993). Untreated normal BALB/c or C3H mice were used to determine normal levels of serum antibodies and treated BALB/c mice to demonstrate what steroid treatment does to suppress normal antibody levels as a measure of side effects.

ELISA plates (Costar, medium binding) were coated with the antigen target proteins (Sigma) and incubated overnight at 4° C. With the exception of cardiolipin, all proteins were diluted in 0.05 M carbonate buffer pH 9.6. The plates were coated with 1 μg/well of collagens II and IV, and laminin, and 0.5 μg/well for all heat shock proteins and heparan sulfate proteoglycan. Plates were also coated with carbonate buffer alone as a control for background binding to the ELISA plates. Cardiolipin was dissolved and diluted in 95% ethanol, plates coated with 1.5 μg/well, and left uncovered overnight at 4° C. to allow solvent to evaporate. Wells with 95% ethanol were used as controls.

After 18 hours ELISA plates were washed 3× with 200 μl/well wash buffer (PBS, 0.05% Tween 20, pH 7.2-7.4) and blocked with 300 μl/well wash buffer containing 3% BSA for one hour. All incubations were done at room temperature on a shaker. The plates were then washed 3× with wash buffer and incubated with 200 μl/well of mouse serum at multiple dilutions with wash buffer containing 1% BSA. Sera from autoimmune and normal mice (1:50 dilutions) were incubated separately both with wells containing the protein antigen and wells containing buffer alone. Wells with no serum were also included as controls. Each condition was run in quadruplicate. After a two hour incubation, plates were washed 3× and incubated with 200 μl/well of 1:3000 dilution of anti-mouse IgG, alkaline phosphatase conjugated (Sigma) for two hours. Alkaline phosphatase yellow (PNPP) liquid substrate system for ELISA (Sigma) is added according to the manufacturer's instructions. The optical density (OD) at 405 and 450 nm wavelength is determined using a Molecular Devices SpectraMax Plus using SoftMax Pro software. The 450 nm reading was subtracted from the 405 reading to correct for optical imperfections in the plate. The OD readings from the wells containing mouse sera but no antigen represented background binding to the ELISA plate and were subtracted to derive the most accurate reactivity due to antigen presence. The four replications for normal and autoimmune mice were compared statistically with t-tests for each antigen.

Cochlear homogenates were also used to coat wells and serum from the mice overlaid to determine the level of circulating autoantibodies against cochlear tissues. All other steps are the same as above.

There were significantly greater antibody levels against these antigens in the autoimmune mice when compared to sera from normal mice. These findings demonstrate that the circulating antibodies in autoimmune mice recognize antigens reported for hearing disorders. This method will allow one to determine which systemic autoantibodies are responsive to steroids. If the glucocorticoid function is to suppress immune disease, then it should facilitate steroid responsive hearing loss by suppressing these systemic autoantibodies.

### EXAMPLE 10

### Mineralocorticoid Receptor Activated Gene Products

The ELISA technique described above in Example 9 can be used to detect the amount of Na⁺ channels and Na⁺K⁺-ATPase and how this is altered in steroid therapy. Wells are coated with these commercially available antigens and the cochlear homogenates are overlaid to capture these proteins within the ear. Detection antibodies are applied to label the channel and enzyme proteins. An antibody that recognizes all isoforms of Na⁺,K⁺-ATPase can be used to determine if this enzyme group as a whole is upregulated in the cochlea by steroids. These products are examined to determine the relative impact steroids are having on the mineralocorticoid receptor mediated activity.

### EXAMPLE 11

### Steroid Receptor and Na⁺,K⁺-ATPase Distribution in the Ear

This example describes immunohistochemical methods used to compare localization, density, and number of mineralocorticoid and glucocorticoid receptors, as well as Na⁺,K⁺-ATPase molecules, following steroid treatment.

Frozen sections of ears from normal BALB/c mice and MRL/MpJ-Fas^{lpr} autoimmune mice were prepared, and subsequently incubated with antibodies to detect the presence of the mineralocorticoid and glucocorticoid receptors, as well as the Na⁺,K⁺-ATPase. Following decalcification in EDTA, the ears were cryostat sectioned following the methods described in Example 4 for improved OCT compound infiltration and tissue preservation. Kidneys were used as positive controls and ear sections without primary antibody as negative controls. Evaluation of the control and autoimmune ears was made to correlate the presence of antibodies with areas of degeneration and recovery with steroid treatment. Staining patterns of the antibodies in the stria vascularis, hair cells, spiral ganglion, and vessels within the other regions of the cochlea, were determined. All steroid receptors were found in the inner ear as well as Na⁺,K⁺-ATPase. Thus the ear has receptors necessary for steroid control of hearing.

### EXAMPLE 12

### Steroid Control of Autoimmune Hearing Loss

This example describes methods that can be used to establish the lowest effective dose of fludrocortisone in the presence or absence of glucocorticoid (such as prednisone) for control of inner ear function and systemic autoimmune disease. Similar methods can be used in humans. Both mineralocorticoid and glucocorticoid treatment control hearing loss in a dose dependent manner, while only the glucocorticoid will control systemic autoimmune disease (see Example 6). As shown above, combining mineralocorticoid and glucocorticoid effectively controls hearing loss and systemic disease at lower doses than either alone.

Mice are treated with successively lower doses of fludrocortisone in the presence or absence of successively lower doses of prednisolone, to characterize their control of autoimmune hearing dysfunction and manifestations of systemic autoimmune disease. This will determine the lowest dose of both steroids that effectively prevent cochlear dysfunction and systemic autoimmune disease. This also will determine new cochlear recovery mechanisms driven by the steroid receptors. These are the glucocorticoid receptor function of suppressing the immune system (cochlear specific autoantibodies) and the mineralocorticoid receptor driven upregulation of sodium transport (Na channels and Na⁺, K⁺-ATPase).

Steroid Treatments

Prednisolone was administered at 1, 3, 5, and 10 mg/kg/day as described in Example 1. Prednisolone alone at 3, 5 and 10 mg/kg/day was effective in preventing (reversing) hearing loss and systemic autoimmune disease, but 1 mg/kg/day prednisolone was not. The higher dose of 10 mg/kg/day was slightly more effective in controlling systemic disease.

Fludrocortisone is administered at 0.5, 1, 3, 5, 10, 15, 30, and 50 μg/kg/day, in the presence or absence of prednisolone as described in Example 1. The highest dose of fludrocortisone alone that was not effective in reversing or stabilizing hearing loss would subsequently be combined with 0.5 or 1 mg/kg/day prednisolone to determine if the combination of fludrocortisone and prednisolone is effective, even though these doses alone are not effective. These doses are administered to autoimmune mice (n=20 each group) to determine their relative treatment effects.

Baseline (2 month old mice) auditory brainstem response audiometry (ABR) is measured and serum samples collected prior to treatment as described in Example 2. An untreated sample (n=20) of water controls will establish the usual progression of cochlear pathology and systemic autoimmune disease. Each steroid dose also will be given to BALB/c normal mice (n=20 each group) to establish steroid effects on the normal cochlea and quantify side effects on the systemic areas of interest. After two-four months of treatment, the following analyses are made to determine steroid driven responses.

Combination treatments will be given to autoimmune mice based on the lowest effective doses determined above. The relative amount of each drug given is successively reduced by half in progressive treatments until no effect on cochlear function or systemic disease is observed. For example, the lowest effective dose was determined to be 3 mg/kg/day of prednisolone (P). The lowest effective dose for fludrocortisone is not known, but assuming it is 10 μg/kg/day (F), the first treatment (n=20 mice) will be P-3 mg/F-0 μg. If this is effective in controlling the auditory and systemic measures, then the next group tested will be given P-1.5 mg/F-5 μg. This 50% reduction in dose will be continued until no effect is seen. As above, an untreated sample (n=20) of water controls will establish the usual progression of cochlear pathology and systemic autoimmune disease, and the effects of each steroid combination will be determined on BALB/c normal mice (n—20 each group) to establish any potential side effects. Baseline (2 month old mice) auditory brainstem response audiometry (ABR) will be measured and serum samples will be collected prior to treatment. After 2 months of treatment, the same cochlear and serum analyses as above will be made on the mice.

It is possible that one combination will no longer have a systemic immune effect while still having a cochlear effect, or vice versa. If this is the case, then the lowest dose of prednisolone for a positive systemic autoimmune effect will be matched with the lowest dose for fludrocortisone for a cochlear effect to determine the final endpoint of treatment.

Cochlear Anatomy and Physiology

ABR is measured on all ears as described in Example 2, to determine levels of cochlear function. The posttreatment ABR is used to assess shift (or lack) in thresholds over the treatment period. Endocochlear potential (EP) is measured following ABR analyses, as described in Example 7.

Subsequently, tissues are collected for cochlear and systemic analyses. Mice are bled for a posttreatment serum analysis and then sacrificed for tissue collection. Fixative is intracardially perfused in 15 mice. The left ears from 10 mice are used for frozen section immunohistochemistry and the right ears from the same 10 are used for quantitative morphology described in Example 4. The other 5 mice have both ears embedded for electron microscopy described in Example 4. Both ears from the remaining 5 mice are collected unperfused and used in the analysis of cochlear specific antibodies and gene products described in Example 9.

Systemic Autoimmune Disease

The glucocorticoids mediate suppression of the immune system, which leads to lowered systemic immune complexes (total serum immunoglobulin) and other general systemic disease factors. These general disease features are helpful in assessing the overall status of systemic disease. Also, these measures change detrimentally in normal mice, so they allow one to assess the side effects of steroid treatment as well. Therefore, these general systemic measures are performed on all mice in addition to very specific antibody detections by ELISA below.

Serum from baseline and posttreatment sampling is analyzed for circulating levels of immune complexes (total immunoglobulin), anti-nuclear antibodies, and hematocrit as described in Example 3. Body weights are determined at each ABR session and spleens are weighed at the termination of treatment. Hematocrits decrease in autoimmune disease due to anti-erythrocyte antibodies. Steroid treatments cause these levels to recover, but in normal mice hematocrits increase above normal due to stimulation of erythropoietic stem cells. Body weight increases with autoimmune disease, but is reversed with steroid treatment. Also, spleen weights increase with autoimmune disease, but remain normal with steroid treatments. Body and spleen weight reduction, along with hematocrit increase, are all severe side effects of glucocorticoid treatment seen in normal mice Therefore, these side effects are examined in normal mice given the steroid treatments.

Cochlear Autoantibodies

Glucocorticoids suppress the immune system and lower immune complexes, which are likely to benefit the ear indirectly. If autoantibodies against the cochlea cause the hearing loss, then decreasing anti-cochlear antibodies with steroid treatment would be a beneficial steroid responsive mechanism. Therefore, it is helpful to determine if this suppression of this systemic autoimmunity is beneficial to cochlear cellular processes. The ELISA technique can be used to quantify the levels of serum autoantibodies against the various putative cochlear antigens (collagen II and IV, heat shock protein 70, laminin, heparan sulfate, and cardiolipin) as described in Example 9. Untreated autoimmune mice and treated normal BALB/c mice provide controls. Steroid treated BALB/c mice show what treatment does to normal antibody levels to determine potential side effects from immune suppression.

To determine if any of these antibodies specifically affect cochlear tissues, both cochleas from the unperfused 5 mice in each group are homogenized (see Trune et al., Hear. Res. 105:57-64, 1997) and reactivity of serum antibodies against these tissues determined by ELISA as described in Example 9. Serum autoantibodies and cochlear homogenates are run in parallel to compare systemic and cochlear specific levels. It is believed that mineralocorticoid treatment will not affect the glucocorticoid receptor and systemic autoimmune disease.

Mineralocorticoid Receptor Activated Gene Products

Mineralocorticoid receptor binding activates genes responsible for production of Na channels and Na⁺,K⁺-ATPase. These channels and enzymes are located in the ear, along with the mineralocorticoid receptor. The levels of these factors can be determined in the same homogenized cochleas by ELISA using the methods described in Example 9. These products are examined in glucocorticoid and/or mineralocorticoid treatments to determine the impact both steroids have on mineralocorticoid receptor mediated activity.

These studies provide a comprehensive assessment of steroid impact on inner ear function (ABR, EP), inner ear morphology (stria measurements), systemic and cochlear autoantibodies (immune complexes, ELISA); and upregulated gene products (immunohistochemistry, ELISA). The results from these studies will provide significant new findings regarding the cellular and molecular mechanisms of the ear that are under the control of steroid responsive mechanisms, as well as explore alternative steroid therapies that may be more effective than those currently employed.

If one low dose of glucocorticoid is effective for systemic disease control, but not auditory control, the lowest dose will be used that provides effective results for both.

### EXAMPLE 13

### Middle Ear Steroid Application

This example describes methods that can be used to administer steroids to the middle ear to control hearing loss. Injection of steroids, either alone or in combination, into the middle ear space may effectively control cochlear dysfunction. An advantage to middle ear delivery is that it should lessen toxicity systemically.

Previous studies describe a method for injecting steroids directly into the middle ear to help cochlear disease processes (Parnes et al., Laryngoscope Suppl. 91:1-17, 1999; Chandrasekhar et al., Otolaryngol. Head Neck Surg. 122:521-8, 2000; Silverstein et al., ENT-Ear Nose Throat J 75:468-88, 1996). This middle ear injection approach avoids the systemic effects of steroid treatment and increases the amount of steroid entering the inner ear compared to systemic injections. However, these experiments were performed on normal animals, and no effect of the steroid on steroid-responsive cochlear disease was reported. Therefore, the control of autoimmune hearing loss in the mice described in Example 1 can be determined to compare the relative efficacies of the mineralocorticoid and glucocorticoid separately and in combination.

Previous studies in guinea pigs showed that glucocorticoid doses were comparable to the oral delivery treatments (0.5 to 20 mg/kg) disclosed herein. The injection volume of 100 μl was used in the guinea pig based on middle ear volume; to adjust that for mice, a volume of 25 μl is used. In humans, a volume of about 100-200 μl can be used. It is possible to obtain an effective dose into that volume. The fluid can be left in the middle ear for longer periods of time (>8 hours, such as at least one day, such as 5-7 days) and retest at one week. The fluid should be reabsorbed by that time. In experimental subjects, the opposite ear is injected with vehicle only to test cochlear function for any potential auditory processing effects of fluid.

### EXAMPLE 14

### Method for Generating Mimetics

Compounds or other molecules which affect mineralocorticoid receptor function, such as compounds which bind to the same site on mineralocorticoid receptor as does fludrocortisone, and also treat hearing loss (by at least restoring some hearing, and/or reducing progression of hearing loss) can be identified and/or designed. These non-antibody compounds or molecules are known as mimetics, because they mimic the biological activity of fludrocortisone. The following example is described with respect to fludrocortisone, but similar techniques can be applied to find mimetics that affect the function of other agents that affect mineralocorticoid receptor function.

Crystallography

To identify which amino acids of the mineralocorticoid receptor interact with fludrocortisone, fludrocortisone is co-crystallized in the presence of the mineralocorticoid receptor. One method that can be used is the hanging drop method. In this method, a concentrated salt, fludrocortisone and the mineralocorticoid receptor solution is applied to the underside of a lid of a multiwell dish. A range of concentrations may need to be tested. The lid is placed onto the dish, such that the droplet “hangs” from the lid. As the solvent evaporates, a crystal is formed, which can be visualized with a microscope. This crystallized structure is then subjected to X-ray diffraction or NMR analysis which allows for the identification of the amino acid residues of the mineralocorticoid receptor that are in contact with fludrocortisone. The amino acids that contact fludrocortisone establish a pharmacophore that can then be used to identify drugs that interact at that same site.

Identification of drugs

Once these amino acids have been identified, one can screen synthetic drug databases (which can be licensed from several different drug companies), to identify drugs that interact with the same amino acids of mineralocorticoid receptor that fludrocortisone interact with. Moreover, structure activity relationships and computer assisted drug design can be performed as described in Remington, The Science and Practice of Pharmacy Chapter 28.

After synthetic drugs or peptides that bind to the mineralocorticoid receptor have been identified, their ability to treat hearing loss can be tested as described in the Examples herein. Those that are positive would be good candidates for therapies for subjects suffering from hearing loss.

### EXAMPLE 15

### Pharmaceutical Compositions and Modes of Administration

This example provides methods and pharmaceutical compositions that can be used to administer fludrocortisone or a mimetic thereof (alone or in combination with other therapeutic agents). Administration of such compositions to a subject can begin whenever treatment of symptoms associated with hearing loss is desired. While compositions that include fludrocortisone or a mimetic thereof are typically be used to treat human subjects, they can also be used to treat similar or identical diseases in other vertebrates such as other primates, farm animals such as swine, cattle and poultry, and sport animals and pets such as horses, dogs and cats.

The pharmaceutical compositions that include fludrocortisone or a mimetic thereof can be formulated in unit dosage form, suitable for individual administration of precise dosages. A therapeutically effective amount of fludrocortisone or a mimetic thereof can be administered in a single dose, or in multiple doses, for example daily, during a course of treatment. Compositions that include fludrocortisone or a mimetic thereof can be administered whenever the effect (such as decreased symptoms of hearing loss) is desired. A time-release formulation can also be utilized.

A therapeutically effective amount of a composition that includes fludrocortisone or a mimetic thereof can be administered as a single pulse dose, as a bolus dose, or as pulse doses administered over time. In pulse doses, a bolus administration of a composition that includes fludrocortisone or a mimetic thereof is provided, followed by a time-period wherein no fludrocortisone or a mimetic thereof is administered to the subject, followed by a second bolus administration. In specific, non-limiting examples, pulse doses of compositions that include fludrocortisone or a mimetic thereof are administered during the course of a day, during the course of a week, or during the course of a month.

The therapeutically effective amount of a composition including fludrocortisone, or a mimetic thereof can depend on the molecule utilized, the subject being treated, the severity and type of the affliction, and the manner of administration, and should be decided according to the judgment of the practitioner and each subject's circumstances. Therapeutically effective amounts of compositions that include fludrocortisone or a mimetic thereof, are those that rescue hearing loss by a desired level, or that stabilize hearing loss, or both. In vitro assays can be employed to identify optimal dosage ranges. Effective doses can be extrapolated from dose-response curves derived from in vitro or animal model test systems. For example, a therapeutically effective amount of fludrocortisone, or a mimetic thereof, can vary from about 0.001 μg per kilogram (kg) body weight to about 20 mg per kg body weight, such as about 1 μg to about 5 mg per kg body weight, such as about 2 μg to about 0.5 mg per kg body weight, or about 5 μg to about 1 mg per kg body weight. The exact dose is readily determined by one of skill in the art based on the potency of the specific compound (such as fludrocortisone or a mimetic thereof) utilized, the age, weight, sex and physiological condition of the subject.

The compositions or pharmaceutical compositions can be administered by any route, including intravenous, intraperitoneal, subcutaneous, sublingual, transdermal, intramuscular, oral, topical, intraotically (including the middle ear), intratympanic, transtympanically, transmucosal, or by pulmonary inhalation. Compositions useful in the disclosure may conveniently be provided in the form of formulations suitable for parenteral (including intravenous, intramuscular and subcutaneous), nasal, intraotically, intratympanically, transtympanically, or oral administration. The term “parenteral” refers to modes of administration which include intravenous, intramuscular, intraperitoneal, intrastemal, subcutaneous and intraarticular injection and infusion.

In one example, pharmaceutical compositions disclosed herein are delivered locally to the area in need of treatment, for example, by local injection into the ear, such as the middle ear, topical application (such as by administration of drops into the ear), or by administration of a transdermal patch near or on the ear. Furthermore, the pharmaceutical compositions or methods of treatment can be administered in combination with other therapeutic treatments, such as other agents that restore hearing or stabilize hearing loss.

In some examples compositions that include fludrocortisone or a mimetic thereof are administered in combination with a therapeutically effective amount of one or more other therapeutic agents, such as other steroids (for example a glucocorticoid, for example a corticosteroid such as prednisolone) or other agents that alleviate hearing loss or other symptoms associated with an immune disorder, such as a glucocorticoid, in a single composition or solution for administration together. In other cases, it may be more advantageous to administer the additional agent separately from fludrocortisone (or a mimetic thereof). Compositions that include fludrocortisone or a mimetic thereof can be administered simultaneously with the additional agent(s), or administered sequentially. In one example, a composition that includes fludrocortisone or a mimetic thereof is formulated and administered with a glucocorticoid as a single dose.

Fludrocortisone or a mimetic thereof can be provided as parenteral compositions, such as for injection or infusion. Such compositions are formulated generally by mixing fludrocortisone or a mimetic thereof at the desired degree of purity, in a unit dosage injectable form (solution, suspension, or emulsion), with a pharmaceutically acceptable carrier, for example one that is non-toxic to recipients at the dosages and concentrations employed and is compatible with other ingredients of the formulation. In addition, fludrocortisone or a mimetic thereof can be suspended in an aqueous carrier, for example, in an isotonic buffer solution at a pH of about 3.0 to about 8.0, preferably at a pH of about 3.5 to about 7.4, 3.5 to 6.0, or 3.5 to about 5.0. Useful buffers include sodium citrate-citric acid and sodium phosphate-phosphoric acid, and sodium acetate/acetic acid buffers. The active ingredient, optionally together with excipients, can also be in the form of a lyophilisate and can be made into a solution prior to parenteral administration by the addition of suitable solvents. Solutions such as those that are used, for example, for parenteral administration can also be used as infusion solutions.

A form of repository or “depot” slow release preparation can be used so that therapeutically effective amounts of the preparation are delivered into the bloodstream over many hours or days following transdermal injection or delivery. Such long acting formulations can be administered by implantation (for example subcutaneously or intramuscularly) or by intramuscular injection. The compounds can be formulated with suitable polymeric or hydrophobic materials (for example as an emulsion in an acceptable oil) or ion exchange resins, or as sparingly soluble derivatives, for example, as a sparingly soluble salt.

Fludrocortisone, or a mimetic thereof, can be utilized as free bases, as acid addition salts or as metal salts. The salts ideally are pharmaceutically acceptable, and these will include metal salts, particularly alkali and alkaline earth metal salts, such as potassium or sodium salts. Numerous pharmaceutically acceptable acid addition salts are available. Such products are readily prepared by procedures well known to those skilled in the art.

Pharmaceutical compositions that include fludrocortisone or a mimetic thereof as an active ingredient will normally be formulated with an appropriate solid or liquid carrier, depending upon the particular mode of administration chosen. The product can be shaped into the desired formulation. In one example, the carrier is a parenteral carrier, preferably a solution that is isotonic with the blood of the recipient. Examples of such carrier vehicles include water, saline, Ringer's solution, glycerol and dextrose solution. Non-aqueous vehicles such as fixed oils and ethyl oleate are also useful herein, as well as liposomes. Other carriers include, but are not limited to: fillers, such as sugars, for example lactose, saccharose, mannitol or sorbitol, cellulose preparations and/or calcium phosphates, for example tricalcium phosphate or calcium hydrogen phosphate, also binders, such as starches, for example corn, wheat, rice or potato starch, methylcellulose, hydroxypropyhnethylcellulose, sodium carboxymethylcellulose and/or polyvinylpyffolidone, and/or, if desired, disintegrators, such as the above-mentioned starches, also carboxymethyl starch, cross-linked polyvinylpyrrolidone, alginic acid or a salt thereof, such as sodium alginate. Additional pharmaceutically acceptable carriers and their formulation are described in standard formulation treatises, such as Remington's Pharmaceutical Sciences by E. W. Martin. See also Wang, Y. J. and Hanson, M. A., Journal of Parenteral Science and Technology, Technical Report No. 10, Supp. 42:2S, 1988.

If desired, the disclosed pharmaceutical compositions can also contain minor amounts of non-toxic auxiliary substances, such as wetting or emulsifying agents, preservatives, and pH buffering agents and the like, for example sodium acetate or sorbitan monolaurate. Excipients that can be included in the disclosed compositions include flow conditioners and lubricants, for example silicic acid, talc, stearic acid or salts thereof, such as magnesium or calcium stearate, and/or polyethylene glycol, or derivatives thereof.

Compositions including fludrocortisone or a mimetic thereof can be administered by sustained-release systems. Suitable examples of sustained-release systems include suitable polymeric materials (such as, semi-permeable polymer matrices in the form of shaped articles, for example films, or mirocapsules), suitable hydrophobic materials (for example as an emulsion in an acceptable oil) or ion exchange resins, and sparingly soluble derivatives (such as, for example, a sparingly soluble salt). Sustained-release compositions can be administered orally, parenterally, intracistemally, intraperitoneally, topically (as by powders, ointments, gels, drops or transdermal patch), bucally, intraotically, intratympanically, transtympanically, or as an oral, otic, or nasal spray. Sustained-release matrices include polylactides (U.S. Pat. No. 3,773,919, EP 58,481), copolymers of L-glutamic acid and gamma-ethyl-L-glutamate (Sidman et al., Biopolymers 22:547-556, 1983, poly(2-hydroxyethyl methacrylate)); (Langer et al., J. Biomed. Mater. Res. 15:167-277, 1981; Langer, Chem. Tech. 12:98-105, 1982, ethylene vinyl acetate (Langer et al., Id.) or poly-D-(−)-3-hydroxybutyric acid (EP 133,988).

Sustained-release compositions include liposomes containing fludrocortisone or a mimetic thereof (see generally, Langer, Science 249:1527-1533, 1990; Treat et al., in Liposomes in the Therapy of Infectious Disease and Cancer, Lopez-Berestein and Fidler (eds.), Liss, New York, pp. 317-327 and 353-365, 1989). Liposomes containing fludrocortisone or a mimetic thereof are prepared by known methods: DE 3,218,121; Epstein et al., Proc. Natl. Acad. Sci. U.S.A. 82:3688-3692, 1985; Hwang et al., Proc. Natl. Acad. Sci. USA. 77:4030-4034, 1980; EP 52,322; EP 36,676; EP 88,046; EP 143,949; EP 142,641; Japanese Patent Application No. 83-118008; U.S. Pat. No. 4,485,045, U.S. Pat. No. 4,544,545; and EP 102,324.

Preparations for administration can be suitably formulated to give controlled release of fludrocortisone or a mimetic thereof. For example, the pharmaceutical compositions can be in the form of particles comprising a biodegradable polymer and/or a polysaccharide jellifying and/or bioadhesive polymer, an amphiphilic polymer, an agent modifying the interface properties of the particles and a pharmacologically active substance. These compositions exhibit certain biocompatibility features that allow a controlled release of the active substance. See U.S. Pat. No. 5,700,486.

Compositions that include fludrocortisone or a mimetic thereof can be delivered by way of a pump (see Langer, supra; Sefton, CRC Crit. Ref. Biomed. Eng. 14:201, 1987; Buchwald et al., Surgery 88:507, 1980; Saudek et al., N. Engl. J. Med. 321:574, 1989) or by continuous subcutaneous infusions, for example, using a mini-pump. An intravenous bag solution can also be employed. One factor in selecting an appropriate dose is the result obtained, as measured by the methods disclosed here, as are deemed appropriate by the practitioner. Other controlled release systems are discussed in Langer (Science 249:1527-33, 1990).

In one example, the pump is implanted (for example see U.S. Pat. Nos. 6,436,091; 5,939,380; and 5,993,414). Implantable drug infusion devices are used to provide patients with a constant and long-term dosage or infusion of a drug or any other therapeutic agent. Such device can be categorized as either active or passive.

Active drug or programmable infusion devices feature a pump or a metering system to deliver the drug into the patient's system. An example of such an active drug infusion device currently available is the Medtronic SynchroMed™ programmable pump. Passive drug infusion devices, in contrast, do not feature a pump, but rather rely upon a pressurized drug reservoir to deliver the drug. An example of such a device includes the Medtronic IsoMed™.

For oral administration, the pharmaceutical compositions can take the form of, for example, powders, pills, tablets, or capsules, prepared by conventional means with pharmaceutically acceptable excipients such as binding agents (such as pregelatinized maize starch, polyvinylpyrrolidone or hydroxypropyl methylcellulose); fillers (such as lactose, microcrystalline cellulose or calcium hydrogen phosphate); lubricants (such as magnesium stearate, talc or silica); disintegrants (e.g., potato starch or sodium starch glycolate); or wetting agents (such as sodium lauryl sulphate). The tablets can be coated by methods well known in the art.

For administration by inhalation, the compounds for use according to the present disclosure can be conveniently delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of for example gelatin for use in an inhaler or insufflator can be formulated containing a powder mix of the compound and a suitable powder base such as lactose or starch.

For inhalation, the composition of the present disclosure can also be administered as an aerosol or a dispersion in a carrier. In one specific, non-limiting example, fludrocortisone or a mimetic thereof (alone or in combination with other therapeutic agents or pharmaceutically acceptable carriers), is administered as an aerosol from a conventional valve, such as, but not limited to, a metered dose valve, through an aerosol adapter also known as an actuator. A suitable fluid carrier can be also included in the formulation, such as, but not limited to, air, a hydrocarbon, such as n-butane, propane, isopentane, amongst others, or a propellant, such as, but not limited to a fluorocarbon. Optionally, a stabilizer is also included, and/or porous particles for deep lung delivery are included (e.g., see U.S. Pat. No. 6,447,743).

In the disclosed method of treating or stabilizing hearing loss, the method includes administering to a subject having sensorineural cochlear hearing loss a therapeutically effective amount of fludrocortisone or a mimetic thereof. Fludrocortisone or a mimetic thereof can be administered in a single or divided dose. Suitable single or divided doses include, but are not limited to about 0.5, 1, 3, 5, 10, 15, 30, or 50 μg/kg/day.

The disclosure also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration. Instructions for use of the composition can also be included.

The disclosure provides compositions that include fludrocortisone or mimetics thereof, for example a composition that includes at least 50%, for example at least 90%, of fludrocortisone or a mimetic in the composition. Such compositions are useful as therapeutic agents when constituted as pharmaceutical compositions with the appropriate carriers or diluents.

In view of the many possible embodiments to which the principles of this disclosure may be applied, it should be recognized that the illustrated embodiments are only particular examples of the disclosure and should not be taken as a limitation on the scope of the disclosure. Rather, the scope of the disclosure is in accord with the following claims. I therefore claim all that comes within the scope and spirit of these claims.

I claim:

1. A method for treating sensorineural cochlear hearing loss in a subject, comprising administering a therapeutically effective dose of a composition comprising fludrocortisone or a mimetic or analog thereof to the subject, wherein the composition comprising fludrocortisone treats the hearing loss in the subject.

2. The method of claim 1, wherein the subject has an autoimmune disease that results in hearing loss.

3. The method of claim 2, wherein the autoimmune disease is Wegener's granulomatosis, polyarteritis nodosa, or systemic lupus erythematosus.

4. The method of claim 1, wherein the subject has a sudden and idiopathic hearing loss.

5. The method of claim 1, wherein the subject has endolymphatic hydrops or Meniere's disease.

6. The method of claim 1, wherein the fludrocortisone is fludrocortisone acetate.

7. The method of claim 1, wherein the hearing loss is a reduction in hearing in the subject by at least 20% as compared to hearing in a normal ear.

8. The method of claim 7, wherein the hearing loss is a reduction in hearing in the subject by at least 50% as compared to hearing in a normal ear.

9. The method of claim 1, wherein the subject has hearing loss in one ear.

10. The method of claim 1, wherein the subject has hearing loss in both ears.

11. The method of claim 1, wherein the hearing loss is caused by abnormal sodium-potassium imbalance in endolymph of a stria vascularis of the subject.

12. The method of claim 1, wherein administration of the composition decreases sodium-potassium imbalance in an endolymph of a stria vascularis of the subject.

13. The method of claim 1, wherein administration of the composition increases sodium transport in a stria vascularis.

14. The method of claim 13, the sodium transport increases by at least 10% in the stria vascularis.

15. The method of claim 1, wherein administration of the composition comprising fludrocortisone increases sodium and potassium transport in a stria vascularis.

16. The method of claim 1, wherein the composition comprising fludrocortisone further comprises a pharmaceutically acceptable carrier.

17. The method of claim 1, wherein the method further comprises administering a glucocorticoid to the subject.

18. The method of claim 17, wherein the composition comprising fludrocortisone further comprises the glucocorticoid.

19. The method of claim 17 or 18, wherein the glucocorticoid is prednisone.

20. The method of claim 19, wherein the prednisone is administered at a dose of about 60-800 μg/kg/day.

21. The method of claim 1, wherein the composition is administered orally.

22. The method of claim 1, wherein the composition is administered to the middle ear.

23. The method of claim 1, wherein the composition is administered transtympanically

24. The method of claim 1, wherein the administration of the composition increases hearing by at least 10% as compared to hearing prior to administration of the composition.

25. The method of claim 1, wherein the administration of the composition increases hearing by at least 20% as compared to hearing prior to administration of the composition.

26. The method of claim 1, wherein the fludrocortisone is administered at a dose of about 100-200 μg/kg/day.

27. A composition comprising a therapeutically effective amount of fludrocortisone and a glucocorticoid.
